(19) United States
(12) Patent Application Publication
(10) Pub. No.: US 2009/0208051 A1
(43) Pub. Date: Aug. 20, 2009

(54) METHOD AND APPARATUS FOR FLUOROGENIC DETERMINATION OF LEAD CONCENTRATIONS

(75) Inventors: Brett M. Emo, St. Louis, MO (US); Roger D. Lewis, St. Louis, MO (US); Kee-Hean Ong, St. Charles, MO (US); Jason N. Kennedy, St. Louis, MO (US)

Correspondence Address: HUSCH BLACKWELL SANDERS LLP, 720 OLIVE STREET, SUITE 2400, ST. LOUIS, MO, 63101, US

(73) Assignee: Saint Louis University, St. Louis, MO (US)

(21) Appl. No.: 12/370,504

(22) Filed: Feb. 12, 2009

Related U.S. Application Data
(60) Provisional application No. 61/028,095, filed on Feb. 12, 2008.

(51) Int. Cl. G06K 9/00; G01T 1/00; G01N 33/20

(52) U.S. Cl. 382/100; 250/361 C; 250/362; 436/73

(57) ABSTRACT

A method and apparatus for the use of fluorogenic probes for development of a rapid, and cost-effective determination of lead clearance following lead hazard control activities. The invention involves the use of fluorogenic chemosensors for efficient detection of lead in dust on hard surfaces at regulatory levels. The use of methods for lead visualization based on one or more of the fluorogenic type compounds would be highly beneficial to lead abatement projects. The identification of areas of high lead concentration (hotspots) can allow work to be focused on areas that have the greatest impact on clearance. Also, a rapid and cost-effective means of determining if lead clearance has been met can improve compliance with lead abatement activities. This would decrease the amount of time and materials spent in general surface cleaning and allow the elimination of isolated areas of lead concentration that may otherwise have been missed.

### CROSS REFERENCE TO RELATED APPLICATION

This application is a non-provisional of and claims priority to U.S. Provisional Patent Application Ser. No. 61/028,095, filed Feb. 12, 2008, which document is hereby incorporated by reference herein to the extent permitted by law.

### BACKGROUND OF INVENTION

1. Field of Invention

This invention relates generally to lead abatement detection techniques and, more particularly, to portable detection techniques for lead abatement in a residence.

2. Background Art

People have used lead for different purposes for many years. Lead has been used for soldering pipes, in crystal glassware, in paint mixtures, and many other applications. The hazards of lead poisoning have been known, but only in relatively recent times has the extent of the threat to children moved to the forefront. The ingestion of lead is harmful to people of all ages, but is more damaging to children under six, and unborn fetuses because the developing nervous system is highly susceptible to the toxic effects of the lead. Children exposed to lead can exhibit behavioral and cognitive impairment at low lead exposure levels, with higher lead exposure levels causing anemia, brain damage and other irreversible effects. The risk of lead poisoning to children from lead-based paint was identified as early as 1897.

Children can be exposed to lead in lead-based paint through typical childhood activities, such as sucking and chewing on painted surfaces, ingesting paint chips from damaged areas and putting their hands in their mouth after being contaminated with lead dust. If lead-based paint is removed without appropriate precautions, the airborne particles permeate the area, and can be ingested or inhaled by both children and adults.

Statistics that have been presented indicate that ninety percent of houses built before 1940 contain lead-based paint. Pain used in houses built before 1950 contained as much as 50% lead by dry weight. Lead was commonly used in areas where durability was desire. After the 1940s, the use of lead-based paint decreased in residential homes. It has been estimated that more than 70% of homes built before 1980 have lead in paint and fixtures. It was commonly used in areas where durability was desired, such as trim, cabinets and outdoor areas.

In 1972 the Consumer Products Safety Commission made the first effort to regulate the lead content in paint. The Commission established a maximum lead content in paint at 0.5% lead w/w in residential paint. This limit was considered to be “safe”. In 1977, lead was even further restricted from use in residential paints due to the risk of lead poisoning in children. Any lead content below 0.06% was considered as “lead-free” paint. Any paint with a lead content greater than 0.06% was still considered to be lead-based paint. In 1990, the U.S. Department of Housing and Urban Development (“HUD”) published “Lead-Based Paint: Interim Guidelines for the Identification and Abatement of Lead-Based Paint in Public and Indian Housing.” The HUD guidelines described technical protocols, practices and procedures for testing, abatement, and worker protection in cleanup and disposal of lead-based paint. The HUD guidelines also required inspection of public and Indian housing before 1994, and abatement if the amounts exceeded an action level of 0.5% lead w/w, or 1 mg/cm.sup.2 mass/area concentration.

Although there are no federal requirements for lead abatement in private housing, Title X was passed in 1992 the Residential Lead-Based Hazard Reduction Act, to become effective in 1995. Title X established new requirements for homeowners and Federal agencies, and new actions to improve the safety and effectiveness of lead-based paint identification and remediation activities. This act requires the sellers of homes to disclose the existence of any lead-based paint or hazard in pre-1978 homes, and allow purchasers 10 days to inspect before becoming obligated to purchase the house.

HUD issued new guidelines, entitled “The Guidelines for the Evaluation and Control of Lead-Based Paint Hazards in Housing.” This document provides detailed guidance on identifying lead-based paint and associated hazards in housing, and controlling the hazards safely and efficiently. A significant change made by Title X and the subsequent guidelines was in the working definition of lead-based paint. Lead-based paint hazards now became “any condition that causes exposure to lead from lead-contaminated dust; bare lead-contaminated soil; or lead-based paint that is deteriorated or intact lead-based paint present on surfaces, or impact surfaces that would result in adverse human health effects” in the U.S. Department of Housing and Urban Development's, “The Guidelines for the Evaluation and Control of Lead-Based Paint Hazards in Housing. Government Printing Office”, 1995, p. 1-S. Under this definition, intact lead-based paint was not considered a hazard, but should be monitored and controlled. An exception to monitoring plans was still made for Indian and public housing, where the requirement exists to abate if the housing is modernized.

The requirement of Title X for sellers to disclose the existence of lead-based paint in older homes, based on the HUD guidelines, makes it extremely important to have an inexpensive, yet accurate means of testing the existing paint. Identifying lead-based paint by HUD guidelines can be accomplished by either portable x-ray fluorescence analyzers (XRF) or by laboratory analysis of paint chips. XRFs are expensive to purchase, have radioactive sources, and operators must be trained and licensed. A laboratory analysis is time-consuming, and may also be very costly. Since lead-based paint hazards have gained attention, less costly methods have been developed to identify qualitatively lead-based paint.

Upon detection of lead-based paint, the abatement process requires evacuation of the unit and removal or encapsulation of lead paint. A proper cleanup after abatement is essential since the residual dust is highly toxic. Post-abatement inspection is required prior to re-occupancy. Typically, a damp paper wipe is used to collect dust samples from the abatement site. The samples are then sent to accredited laboratories to measure their lead content.

Two tests that have been developed include sodium sulfide, and a one-step sodium rhodizonate test. These tests have been put into use in spite of their limitations, which include false positives, false negatives, excessive time required for color change, and difficulties seeing the appropriate color change indicating a positive result. The “one-step red” sodium rhodizonate test is actually the first step of a test which has been used in the past for the identification of both barium and lead. Use of the “one-step red” test ignores the previously established limitations of the same procedure. In the past, the results provided by the red color in a positive “one-step red” test indicated the presence of both lead and barium. An additional step was required to differentiate between the two, and for the results to be conclusively interpreted as lead.

Recent years have seen an application of a portion of the sodium rhodizonate test to a new area of interest in lead determination. With the concern regarding the presence of lead in paints used in the past, simple testing methods have become advantageous for use in the field. These tests allow the user to make a qualitative analysis of the lead content in a painted surface. The test results can provide the basis for determining the hazards that may arise from the paint removal, or continued exposure to the painted surface. If a field test is not available, the only alternative is instrumental analysis methods, which require laboratory testing or expensive field instruments. Simple testing kits, using the first step in the sodium rhodizonate test, were patented in the early 1990s. These became commercially available, and were accepted for qualitative lead identification in the field. The results of these tests were often used to decide the hazards of the painted surface and the method for paint removal.

The sodium rhodizonate tests, as are presently in use, largely ignore the interference caused by barium, due to using a test, which historically has been used to detect barium. Additionally, the tests are not completely accurate, may result in false positives when testing for lead, and may require up to 24 hours for completion. The EPA proposed regulation 40 CFR Part 745 (Jan. 10, 2006), proposes the use of a visual comparison of dust collected from a surface to a standard wipe to determine clearance. These methods use the dust wipe as a surrogate for the extent of contamination on a surface, however direct visualization and quantification of lead on a surface, which would be more representative of lead present is not provided. Lead abatement detection techniques have traditionally relied on wipe samples taken from a surface to verify the extent of lead contamination, usually through laboratory testing. Sodium rhodizonate-containing wipes have been useful in determining the presence or absence of lead on a surface but these results are usually not quantitative.

The current practice regarding inexpensive detection of lead after lead abatement activities is the use of sodium rhodizonate. The rhodizonate forms a complex with lead that is calorimetric, and causes a visual pink to red color upon the presence of lead on a surface or on leaded dust. Rhodizonate, however, forms a precipitate with lead and is not ratiometric, or proportionate in response, with a change of lead concentrations, nor can it be used to provide a means of quantifying leaded dust at critical concentrations relevant to health or feasibility based regulations.

The detection of lead-in-paint by x-ray fluorescence spectroscopy (XRF) has become the preferred method in the abatement industry because it is accurate and non-destructive. To measure lead in paint films the technician uses a portable spectrometer that has a source of gamma radiation such as cobalt 57 or cadmium 109, to irradiate the surface being analyzed, and a semiconductor detector, or a scintillation crystal coupled to a photomultiplier as the radiation detector. The technician simply holds the device against the surface for a measured amount of time and a reading is obtained. Unskilled operators can use these machines effectively. Most difficulties encountered with these devices occur when the underlying wall or molding presents an unusual backscatter spectrum, or the overlying non-lead paint layer thickness becomes great enough to cause attenuation of the underlying lead layers.

An x-ray fluorescence analyzer that includes a linear excitation source of radiation, a linear detector, a transmitted radiation trap and a motor driven mechanism to move a sample through the device, built in a compact, portable instrument is capable of reliably analyzing and quantifying microgram concentrations of metallic elements, particularly lead, that is contained in soil or other aggregates, or on a sample medium such as a post abatement dust wipe or an air sampling filter.

Conventional x-ray fluorescence spectrometers can produce quantitative results if either of two conditions are met: the sample is inherently uniform in analyte concentration, or the sample can be prepared such that its volume falls within the linear area of the particular spectrometers field of view. Randomly distributed analyte such as that found in dust wipe or air filter samples cannot usually be quantified without careful sample preparation. X-ray fluorescence spectrometers are inherently non-destructive, and can be made portable, repeatable, quantitative and capable of producing a hard copy result that can be validated.

X-ray fluorescence spectroscopy permits measurement of the atomic composition of materials by observing the radiation emitted by a material when it is excited with a source of high energy photons such as x-rays or gamma rays. X-rays result when an electron is knocked out of its orbit around the nucleus of an atom by a photon from the source. When this occurs, an electron from an outer shell of the atom will fall into the shell of the missing electron. The excess energy in this interaction is expended as an x-ray photon. Since each element has a different and identifiable x-ray signature, the elemental composition of a sample can be identified.

Typically, x-ray fluorescence spectrometers include a source of radiation and a detector. The detector emits electrical impulses that are proportional to the energy of the photons being emitted by the sample. The impulses are amplified and pulses are counted from discrete portions of the sample's spectrum where x-rays emitted by the element under investigation can be found. The data is treated to isolate the x-rays being measured from other nuclear events and electronic noise with the aid of a computer.

Any contribution that can positively affect lead clearance and compliance activities would be welcomed by housing agencies, residents, housing advocates, and public health agencies both nationally and internationally. As a result, it is important to evaluate alternative and improved clearance methods as stipulated in the HUD 2006 Notification of Funds Available (NOFA). There is a need to identify alternative or new technologies that could be used to screen surfaces to indicate if additional cleaning is needed to achieve a clearance value. The improved alternative methods need to be portable and affordable while being accurate and ratio-metric.

### BRIEF SUMMARY OF INVENTION

The present invention is related to chemical sensing, particularly through fluorescence, which demonstrates the utility of fluorogenic probes for specific quantification of lead in biological cells. The invention more specifically is a method and apparatus for the use of fluorogenic and luminescent probes for development of a rapid, and cost-effective determination of lead clearance following lead hazard control activities. The invention involves the use of chemifluorescent probes and in the alternative chemiluminescent probes for efficient detection of lead in dust on hard surfaces at regulatory levels. The use of methods for lead visualization based on one or more of the fluorogenic or luminescent type compounds would be highly beneficial to lead abatement projects. The identification of areas of high lead concentration (hotspots) can allow work to be focused on areas that have the greatest impact on clearance. Also, a rapid, portable and cost-effective means of determining if lead clearance has been met can improve compliance with lead abatement activities. This would decrease the amount of time and materials spent in general surface cleaning and allow the elimination of isolated areas of lead concentration that may otherwise have been missed.

The term chemifluorescence implies a chemical system that is capable of fluorescence. There are two key functional groups that compose a chemifluorescence compound, the fluorophore and a ligand binding site. A fluorophore is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Several factors that can impact the fluorophore are pH, ion concentration, temperature, and the composition of its solvent. Examples of common fluorophores are listed in the appendix, Section A1.

The ligand functional group is responsible for binding a particular element or agent and eliciting an effect which can either turn off (quench) fluorescence or turn on fluorescence. The ligand used for controlling the reaction will vary depending on the application. Specificity for a particular element or agent is an important quality for the ligand functional group, but this is not always possible. For instance, some divalent metals may interact with the same ligand because of valence effects and similar nuclear radii.

The final component of a chemifluorescence (CF) system is the input of excitation wavelength, electromagnetic energy (EM) of a particular wavelength. Each fluorophore has a specific excitation energy range with a peak excitation that drops off on either side to form a roughly bell-shaped curve. This energy input is necessary as photons are absorbed and re-emitted at a lower energy, longer wavelength called the emission wavelength. Much like the excitation wavelength, there will be a wavelength range emitted with a peak. Often the emission curve is significantly skewed. In order to measure this reaction it is common to use a photomultiplier tube or, if the intensity of fluorescence is sufficient, by use of a camera or phosphorescence scanner.

In the alternative, Chemiluminescence (CL) is the reaction of two compounds, a parent compound and an oxidizer, which is catalyzed by an element or agent. When the parent compound reacts with the oxidizer an unstable adduct is formed. Resolution of this instability releases a photon which can then be measured and used to quantify the concentration the catalyst or perform a presence/absence spot test for the element or agent that catalyzes the reaction. An oxidizer is defined as a substance that accepts or gains electrons and, in the process, undergoes reduction. A list of common oxidizers is provided in Appendix section A2. A catalyst is a substance that changes the speed or yield of a chemical reaction without being consumed or chemically changed by the chemical reaction. Catalysts can be organic or inorganic agents. Many metals can act as inorganic catalysts.

Some of the most common demonstrations of the Chemiluminescence phenomena are the male firefly (bioluminescence utilizing the luciferase enzyme), glow sticks, and the use of luminol in crime scene investigations which is oxidized by hydrogen peroxide and catalyzed by the iron found in blood.

CL methods can be utilized in various ways related to metal detection and quantification. For example, the CL compound Lophine can be used and hydrogen peroxide when attempting to measure cobalt, chromium, and copper in solution. The findings indicate problems with this system involving measurement of a mixed sample where measurement of the above metals, individually, did not add up when the equivalent concentrations of these metals were measured collectively. Also, luminol can be used to quantify the concentration of iron in sea water samples. In this study, 18 milliliter sea water samples were collected and applied to a resin column to purify soluble iron prior to analysis using a photomultiplier tube. This dealt with the problems encountered with Lophine by eliminating contaminants that can impact the analysis. Uses of CL compounds can be expanded with applications in the biological sciences for measuring intracellular and extracellular metal ion concentrations. Researchers then coupled the enzymatic activity of a metalloenzyme, alkaline phosphatase, to measure CL in relation to varying trace levels of zinc, beryllium, and bismuth. Then recombinant DNA techniques were used to engineer a plasmid vector which encodes the gene sequence for the synthesis of the bioluminescent compound luciferase (found in fireflies) under the control of the regulation system of the cadA gene which responds to the presence of heavy metals. This vector was transferred into two strains of bacteria, Staphylococcus aureus strain RN4220 and Bacillus subtilis strain BR151. Both strains expressed luciferase in response to cadmium, lead and antimony at nano and micromolar concentrations depending on the strain. There were subsequent studies which used a flow-injection system coupled to a photomultiplier to analyze lead concentration in gasoline using luminol and the oxidizer potassium permanganate. More recently studies have been published with results of a field portable, low cost micro total analytical system for analysis of metal concentrations in environmental water samples.

An example of a potential application of CL technology is the incorporation of a fluorogenic compound in lead abatement detergents can allow a real-time evaluation of cleaning efficacy. Essentially, a surface can be cleaned using a wipe and detergent spray to the point of low or no fluorescence. To accomplish this fluorogenic compounds can be used on environmental surfaces in a home. The fluorogenic probe compounds can “turn-on” at critical lead concentrations that represent clearance values. New technology using fluorogenic probes to detect lead in house dust could speed up the process of declaring clearance or vastly improve lead detection after repair and renovation activities that currently rely on comparison of a wiped cloth to a photomicrograph standard that EPA developed to correlate to a level of contamination that is below the dust lead hazard in 40 CFR 745.65b. However this potential application is not the primary focus or embodiment of the present invention.

The use of fluorogenic probe technology can potentially be used for simple clearance measures, where real-time instrumentation, such as the XRF for wipes or ASV (anodic stripping voltammetry) or off-site laboratory analysis, such as flame atomic absorption or inductively couple plasma (ICP) or other lead analysis, performed by a National Lead Laboratory Accreditation Program lab, is currently used. For example many of the probes identified for Pb²⁺ are not ratiometric and few are water soluble. These characteristics are not necessarily useful for detecting lead in living cells, but a probe that turns on (increases in fluorescence intensity) in the presence of Pb^{z+} is highly desirable for the purposes of environmental sensing, especially if it only turns on in the presence of environmentally relevant concentrations of lead.

Several of the probes not only exhibit an increase in fluorescence emission upon lead binding (Table 2) of FIG. 1, but also have a dissociation constant for lead (10⁻³-10⁻⁶ M) that is in an appropriate range for environmental sensing. Furthermore, for one possible specific application proposed by one embodiment of the present invention (i.e. creating dust wipes that exhibit fluorescence upon exposure to lead), it is particularly desirable to have a sensor that is not soluble in water, so that the sensor can be applied to the wipes using an organic solvent but will not leach out after being dried and when used in the presence of water. Almost all of the small molecule probes reported to date for lead meet these criteria (Table 2). Two of the fluorescent sensors for lead (Leadmium™ Orange and Leadmium™ Green) demonstrate ideal parameters for environmental teat applications.

A probe that has turn on ability but is easily dissolved in a non-toxic solvent, such as water, would enhance the ability to increase extraction efficiency of lead from a dust-wipe . This would accommodate lead on wipes, where a lead gradient or hot spots exists, yet fully solvate lead with a probe that would turn on at a critical level, such as one that is near the clearance level for lead on floors, 40 ug/ft2. After swiping a wipe, the wipe can be placed in a developer solution with probe and the fluorescence emission can be measured. The solution can be shaken vigorously prior taking the measurement. It is also important that a probe be found that has an absorption spectra that can make use of an inexpensive source of light, such as a black lamp.

Yet another potential application of the technology of the present invention, though not the primary focus or embodiment of the invention, is the use of wipes embedded with a probe. The method can be performed by wiping about approximately a 1 ft (+/−4 inches) by 1 ft (+/−4 inches) surface suspected of lead contamination (leaded dust) using a chemosensor embedded dust wipe. After wiping, the surfaces can be illuminated at their excitation frequency by a UV or other appropriate electromagnetic light source and digital pictures can be taken of the wipes one minute after cleaning from a mounted camera at a standard height. The image can be screened for fluorescence emission wavelength using specially designed software and lead concentration can be correlated with average fluorescence intensity of the filtered image. A variation of this embodiment is to utilize the wipe containing the embedded probe in combination with a typical detergent utilized for lead clean up in order to detect if cleanup has been effective.

Yet another embodiment of the present invention is to utilize a method of spraying the chemosensor directly onto a surface for use as a cleaning aid on an about approximately 1 ft (+/−4 inches)×1 ft (+/−4 inches) surface. The surface can be sprayed with a solution containing the chemosensor and allowed to develop for about approximately one minute. A digital picture can be taken of the surface, and average fluorescence can be measured. Average fluorescence intensity can be correlated with lead concentration as determined by analysis with specially designed software. The volume of spray needed to achieve maximum fluorescence can be predefined.

The use of various types of black or LW lamps and bulbs on chemosensor fluorescence can enhance the measurement technique. After the surfaces are sprayed and allowed to develop, and they can then be illuminated with various types of commercially available black lights. The distance from the bulb to surface can be predefined, and emission intensity can be measured using specially designed software. A digital picture of the surface can be taken and screened to measure average fluorescence intensity as described previously. Average fluorescence intensity can be compared against light intensity and against light type.

Yet another variation to the invention is to utilize a sticky backed paper wipe where the wipe is place on the surface with the sticky side making contact with the surface. The wipe can then be peeled off and measurements taken. The sticky surface can have embedded therein with chemosensor or the wipe can be added to a developer solution or blotted with probe and the fluorescence emission can be measured.

The above noted methods can be modified accordingly to utilize a CL or CF chemosensor. Also, the above example applications are further refined in additional embodiments described in the Detailed Description Of Invention section.

Current work in lead abatement has been reliant on wipe samples taken from a surface to verify the extent of lead contamination, usually through laboratory testing and it is time consuming and expensive. New technology using CL or CF probes to detect lead in house dust could speed up the process of declaring clearance or vastly improve lead detection after repair and renovation activities that currently rely on comparison of a wiped cloth to a photomicrograph standard that EPA developed to correlate to a level of contamination that is below the dust lead hazard in 40 CFR 745.65b. CL or CF probes can be developed in accordance with the present invention for a rapid, and cost-effective determination of lead clearance after lead hazard control activities. The present invention can be portable and cost efficient while providing sufficient accuracy and ratio-metric fidelity. These and other advantageous features of the present invention will be in part apparent and in part pointed out herein below.

### BRIEF DESCRIPTION OF THE DRAWINGS

For a better understanding of the present invention, reference may be made to the accompanying drawings in which:

FIG. 1 is an illustration of a chemi-fluorescence solution application;

FIG. 2 is an illustration of a chemi-fluorescence raster image analysis application;

FIG. 3 is an illustration of a chemi-luminescence solution application;

FIG. 4 is an illustration of a chemi-luminescence raster image analysis application;

FIG. 5 is an illustration of a chemi-luminescence light sensitive film methodology;

FIG. 6 is an illustration of a chemi-luminescence surface spray application;

FIG. 7 is an illustration of a chemi-luminescence flow injection system;

FIG. 8 is a graph of the fluorescence response curve of a probe illustrative of ratiometric properties;

FIG. 9 is an illustration of using a sticky back wipe to collect a sample for analysis;

FIG. 10 is an illustration of an alternative process; and

FIGS. 11A-11C is an illustration of the ratiometric relationship attained between lead concentration and fluorescence.

While the invention is susceptible to various modifications and alternative forms, specific embodiments thereof are shown by way of example in the drawings and will herein be described in detail. It should be understood, however, that the drawings and detailed description presented herein are not intended to limit the invention to the particular embodiment disclosed, but on the contrary, the intention is to cover all modifications, equivalents, and alternatives falling within the spirit and scope of the present invention as defined by the appended claims.

### DETAILED DESCRIPTION OF INVENTION

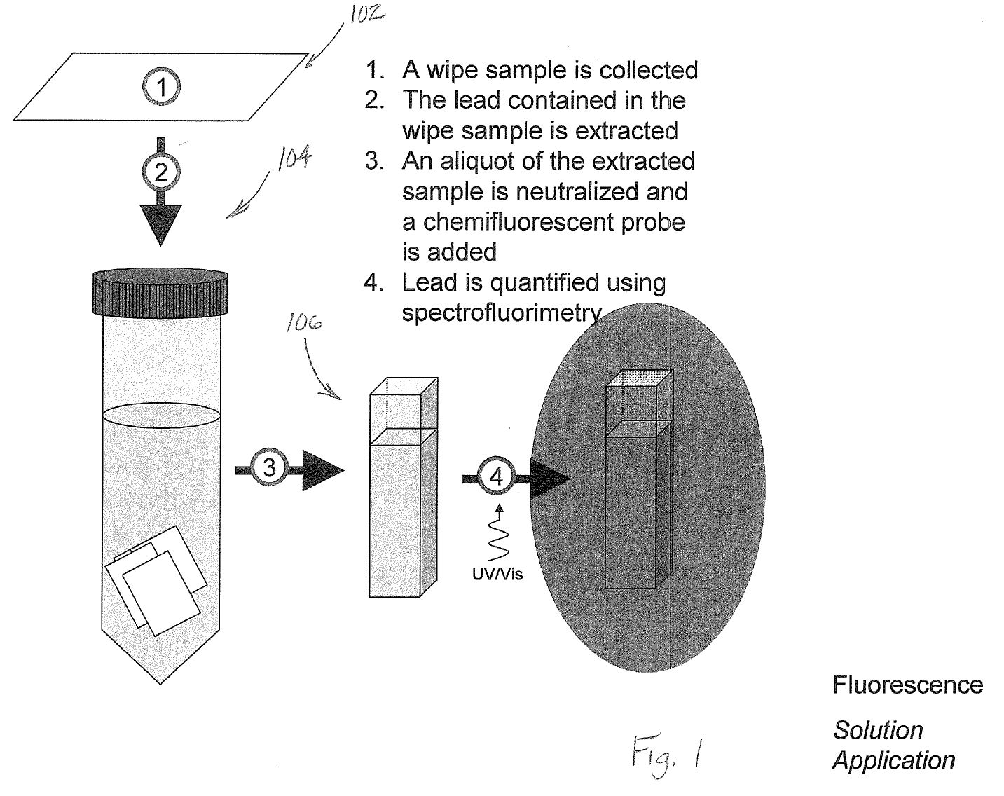

According to the embodiment(s) of the present invention, various views are illustrated in FIG. 1-11 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified.

One embodiment of the present invention comprising the use of a fluorogenic or alternatively a luminescent probe compound to detect the concentration of lead on a surface teaches a novel apparatus and method for detecting the presence of lead in a residential environment. One embodiment of the method for using CL or CF chemosensor probes for detection of lead for lead abatement activities includes wiping a suspect surface with a wipe, then placing the wipe in an extraction solution to extract the lead contained in the wipe, neutralizing an aliquot of the extracted sample and creating a developer solution with a CF probe contained therein. Optionally the method can include the step of agitating said wipe in solution to assist in the extraction of any embedded matter resulting from the wipe action or swipe. As another optional step the user can perform the step of illuminating the solution and capturing an image of the solution, then screening the image for fluorescence emissions indicative of the presence of lead. The developer solution can comprise a fluorogenic chemosensor probe selected from a group of probes consisting of those shown in Appendix A1. If the step of agitating is performed it can be performed at least a minute after the wipe has been placed in solution. If the step of illuminating the solution is performed the solution can be illuminated with a light source having a wavelength appropriate to excite the CF probe. The step of screening the image includes processing the image with software operable to correlate lead concentration with average fluorescence intensity.

Another embodiment of the method for using CL probes for detection of lead for lead abatement activities includes a step of spraying a suspect surface with a developer solution containing a CL probe. An image of the suspect surface can be captured. The image can be screened for emissions indicative of the presence of lead. The CL developer solution can include CL probes having oxidizers as shown in Appendix A2.

Yet another embodiment of the method for using CL or CF probes for detection of lead for lead abatement activities can include placing a sticky side of a sticky back paper wipe against a suspect surface where said sticky side has a slightly tacky adhesive. The sticky side can have a CF or CL developer solution applied to the sticky side and the image of the sticky side can be screened for emissions indicative of the presence of lead.

The step of capturing a digital image and the step of screening the image is can performed with a digital image capturing device

The details of the invention and various embodiments can be better understood by referring to the figures of the drawing. Referring to FIG. 1, an illustration of a CF solution application is shown. One embodiment of the present invention comprising the use of a chemifluorescent probe compound to detect the concentration of lead on a surface teaches a novel apparatus and method for detecting the presence of lead in a residential environment. One embodiment of the method for using chemifluorescent probes for detection of lead for lead abatement activities includes wiping a suspect surface with a wipe 102, placing the wipe in an extraction solution 104, neutralization of the solution, then employing a chemifluorescent probe to quantify the sampled amount of lead 106. Optionally the method can include the step of agitating said wipe in solution (not shown) or the application of heat (not shown) to assist in the extraction of any embedded matter resulting from the wipe action or swipe.

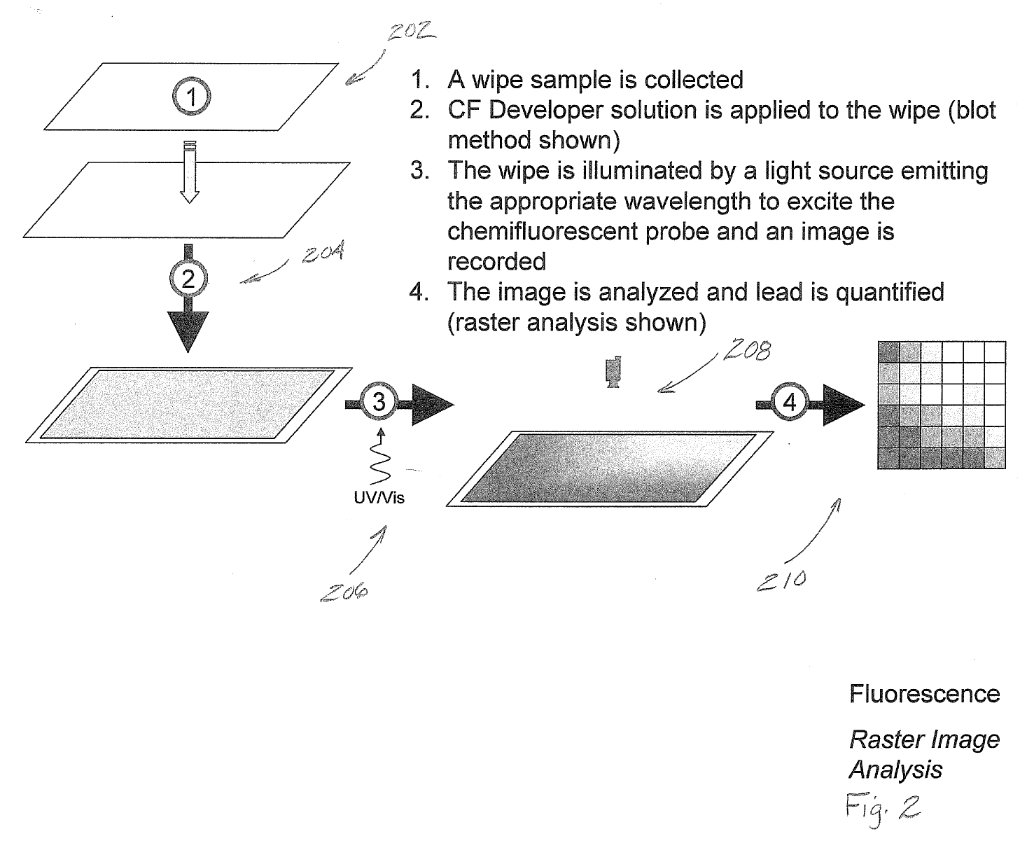

Referring to FIG. 2, an illustration of a CF raster image analysis application is shown. As an alternative the user can collect a wipe sample as previously stated 202, apply a developer solution containing a chemifluorescent probe therein 204, such as for example utilizing a blotting method, illuminate the wipe using light of the appropriate wavelength to cause excitation of the chemifluorescent probe employed 206, then screening the image 208 for fluorescence emission indicative for determining the amount of lead present. The developer solution would have properties that allow for the simultaneous extraction of lead from the wipe with full functionality of the chemifluorescent probe selected from a group of probes consisting of listed in Appendix A1. The step of applying the developer solution to the wipe can be carried out using several methods which include, but are not limited to, spraying the solution on the wipe or blotting the solution on the wipe using a saturated blotting pad. The step of capturing an image of the wipe can be performed using a camera with appropriate illumination or through an imaging system that can scan the wipe and generate an image. The step of screening the image includes processing the image with software to correlate lead concentration with average fluorescence intensity by utilizing such methods as raster image analysis 210.

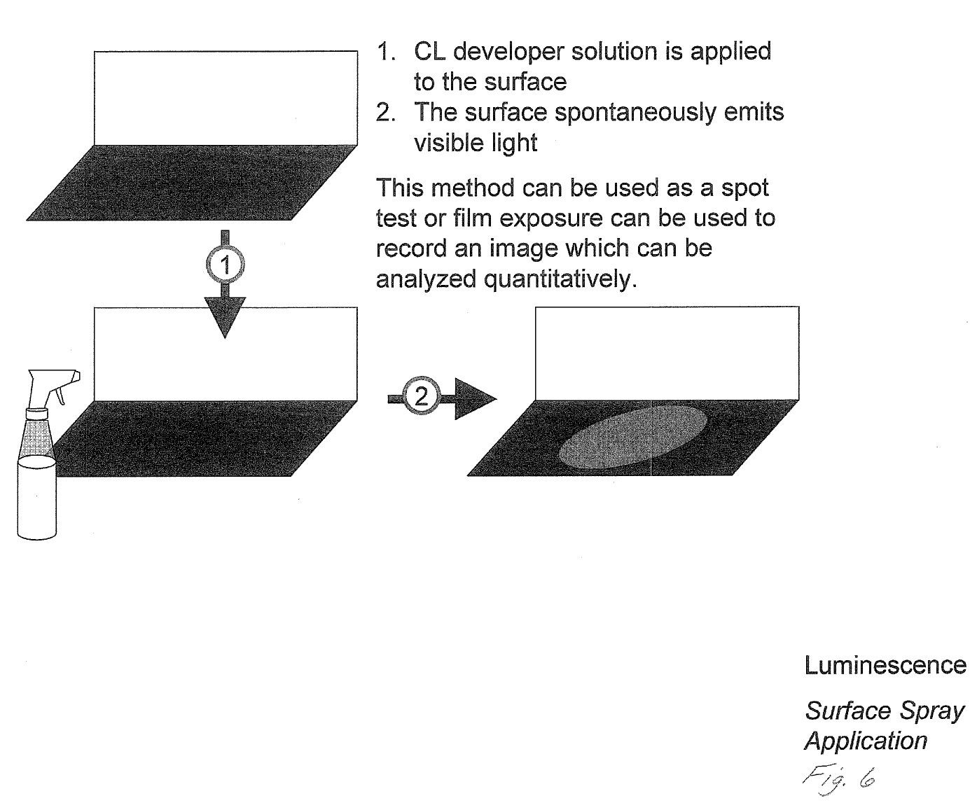

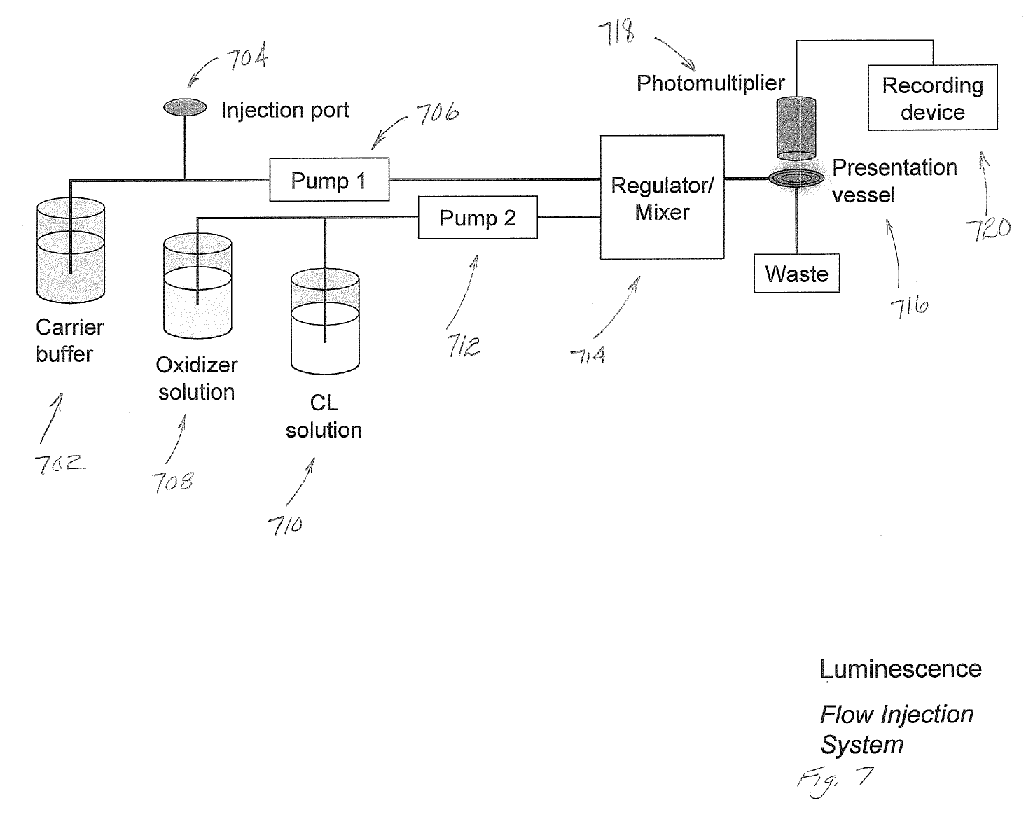

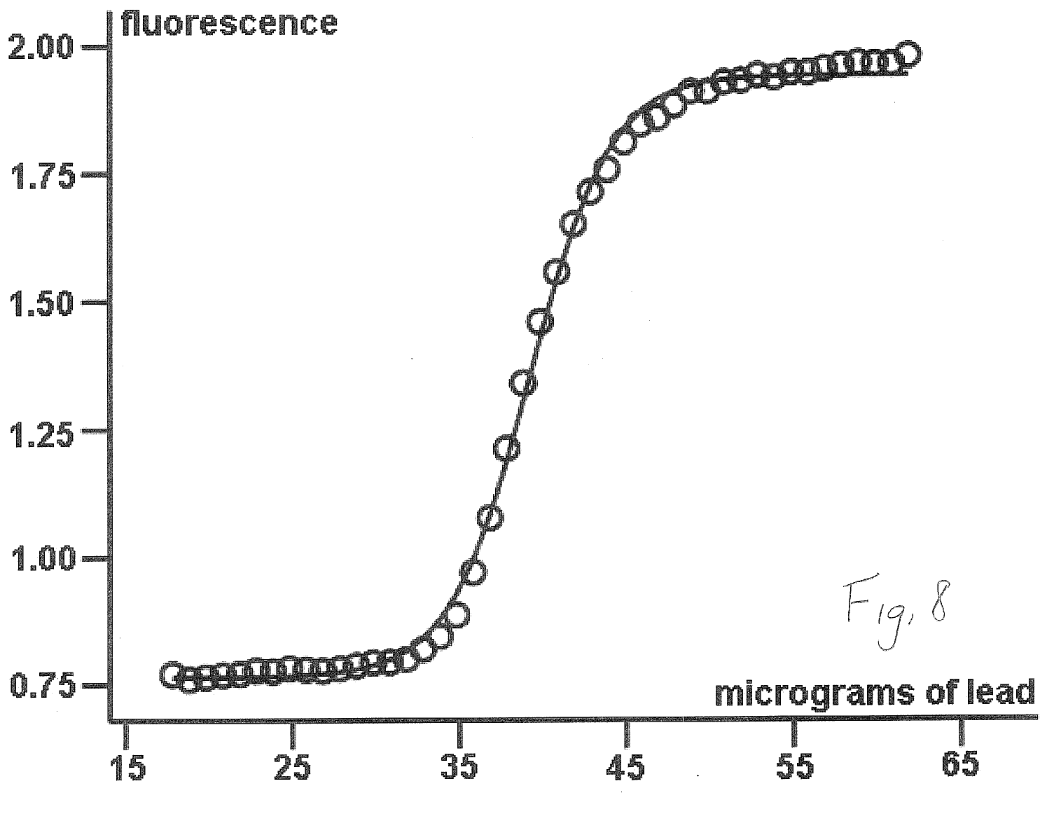

Another embodiment of the method for using chemifluorescent probes for detection of lead for lead abatement activities includes a step of spraying a suspect surface with a developer solution containing a chemifluorescent probe. The suspect surface can be illuminated and an image of the suspect surface can be captured as described previously. The image can be screened for fluorescence emissions indicative of the presence of lead. The step of spraying can be performed with any sprayer capable of delivering an even and diffuse quantity of the solution to the surface. The developer solution can include any detergent compatible with a chemifluorescent probe selected from a group of probes consisting of those shown in Appendix A1. The step of illumination can be performed using any light source which emits light of the appropriate wavelength to cause excitation of the chemifluorescent probe employed. This method is essentially illustrated in FIG. 6, where the CL probe process is shown, however, when using a CF probe there is an additional step of illuminating.

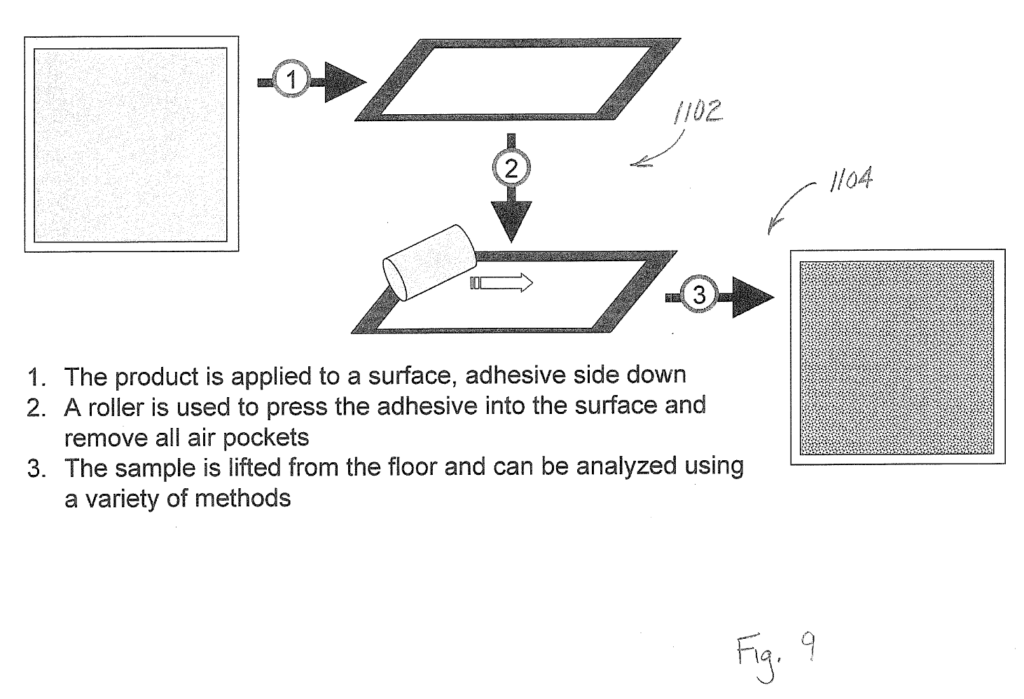

Referring to FIG. 9 and illustration is shown utilizing a sticky back wipe. Yet another embodiment of the method for using chemifluorescent or chemiluminescent probes for detection of lead for lead abatement activities can be the use of a paper or cloth material coated on one side with low tack adhesive which is pressed evenly against the surface to be tested 1102. The material can then be lifted away from the surface, a probe can be applied as described previously and subjected to analysis 1104 in solution or via image processing as described previously. Additionally, the lift sample can be evaluated subjectively as put forth in the EPA proposed regulation 40 CFR Part 745 (Jan. 10, 2006), which uses a visual comparison of dust collected from a surface to a standard to determine clearance. Another alternative would entail capturing an image of the lift sample, analyzing the image by such means as raster analysis, and comparing the average color value to the average color value of a standard to determine clearance. Optionally the paper or cloth material and adhesive can be easily dissolvable to aid extraction of lead into a solution.

The step of capturing an image of the sample can be performed using a camera with appropriate illumination or through an imaging system that can scan the wipe to generate an image. The step of screening the image includes processing the image with software to correlate lead concentration with average fluorescence intensity by utilizing such methods as raster image analysis.

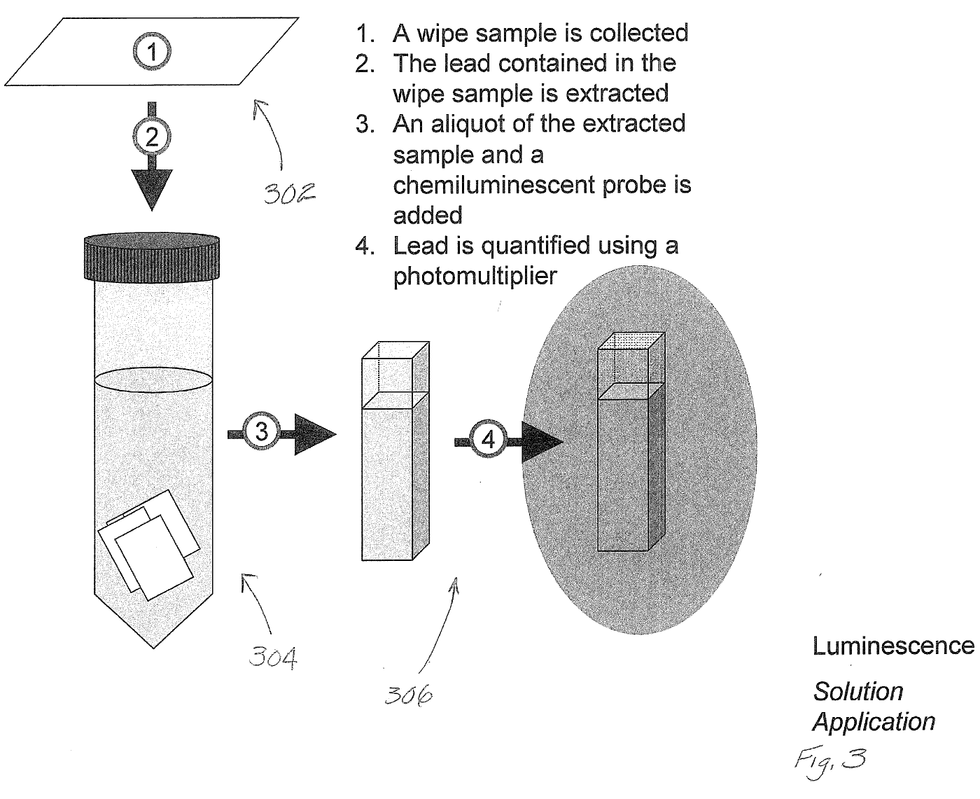

Referring to FIG. 3, an illustration of a CL solution application is shown. One embodiment of the present invention comprising the use of a chemiluminescent probe compound to detect the concentration of lead on a surface teaches a novel apparatus and method for detecting the presence of lead in a residential environment. One embodiment of the method for using chemiluminescent probes for detection of lead for lead abatement activities includes wiping a suspect surface with a wipe 302, placing the wipe in an extraction solution 304, then employing a chemiluminescent probe to quantify the sampled amount of lead 306 using a photomultiplier. Optionally the method can include the step of agitating said wipe in solution (not shown) or the application of heat (not shown) to assist in the extraction of any embedded matter resulting from the wipe action or swipe.

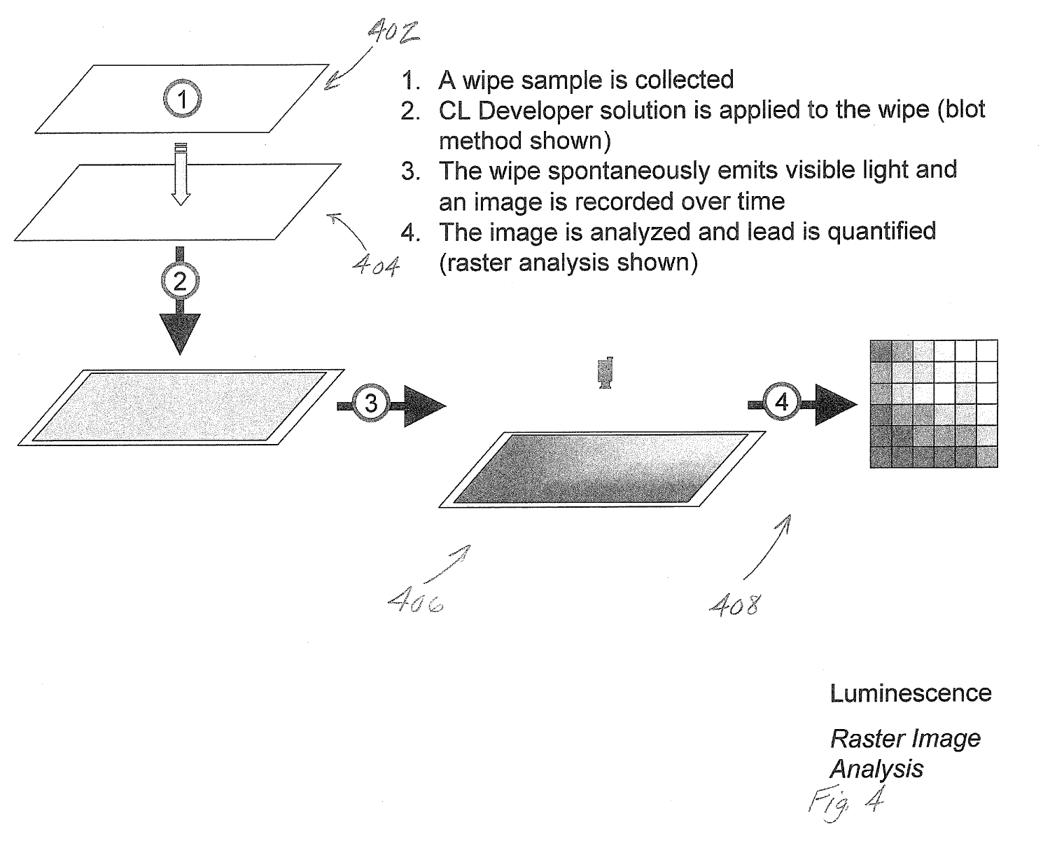

Referring to FIG. 4, a luminescent raster image analysis application is shown. As an alternative the user can collect a wipe sample as previously stated 402, apply a developer solution containing a chemiluminescent probe therein 404, such as for example utilizing a blotting method, then screening the image 406 emission indicative for determining the amount of lead present. The developer solution would have properties that allow for the simultaneous extraction of lead from the wipe with full functionality of the chemiluminescent probe selected from a group of probes consisting of oxidizers listed in Appendix 2. The step of applying the developer solution to the wipe can be carried out using several methods which include, but are not limited to, spraying the solution on the wipe or blotting the solution on the wipe using a saturated blotting pad. The step of capturing an image of the wipe can be performed using a camera with appropriate illumination or through an imaging system that can scan the wipe and generate an image. The step of screening the image includes processing the image with software to correlate lead concentration with average fluorescence intensity by utilizing such methods as raster image analysis 408.

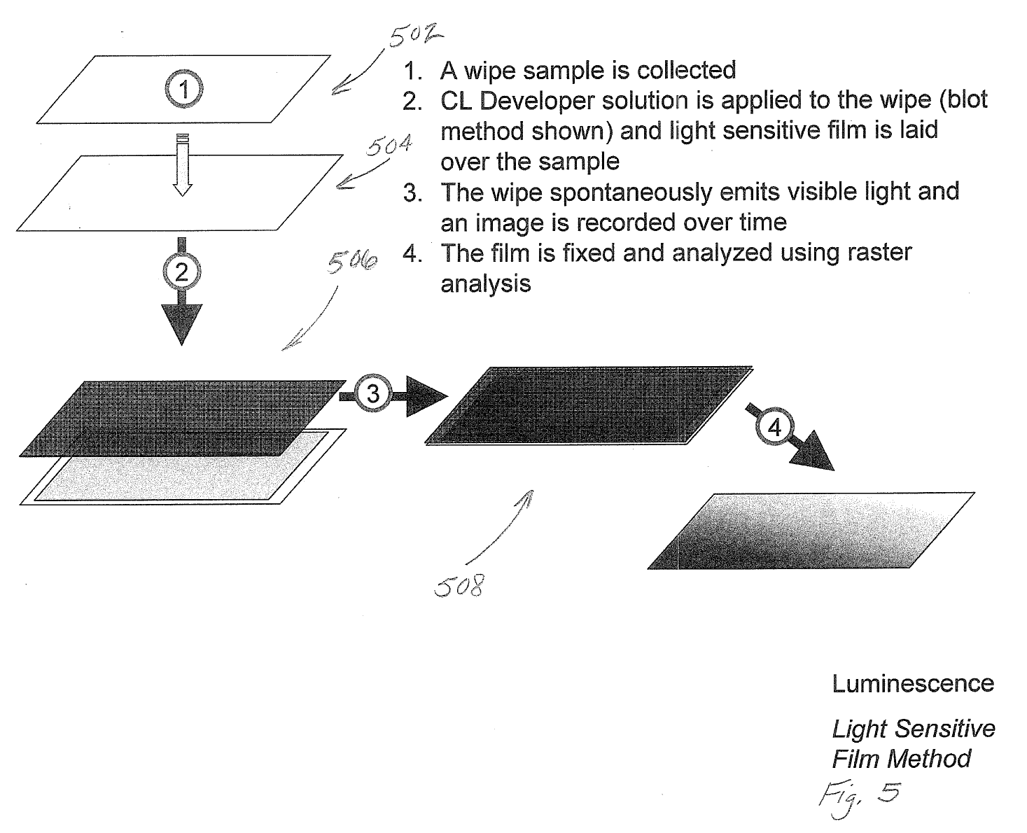

Referring to FIG. 5, an alternative luminescent raster image analysis application is shown. As an alternative the user can collect a wipe sample as previously stated 502, apply a developer solution containing a chemiluminescent probe therein 504, such as for example utilizing a blotting method and laying a light sensitive film over the wipe sample 506, then the wipe spontaneously emits visible light and an image is recorded over time. The emission indicative for determining the amount of lead present. The developer solution would have properties that allow for the simultaneous extraction of lead from the wipe with full functionality of the chemiluminescent probe selected from a group of probes consisting of oxidizers listed in Appendix A2. The step of applying the developer solution to the wipe can be carried out using several methods, which include, but are not limited to, spraying the solution on the wipe or blotting the solution on the wipe using a saturated blotting pad. The step of capturing an image of the wipe can be performed using a light sensitive film appropriate to sense illumination and the resulting fixed film can be analyzed 508 through an imaging system that can scan the film and generate an image. The step of screening the image includes processing the image with software to correlate lead concentration with average fluorescence intensity by utilizing such methods as raster image analysis.

Referring to FIG. 8, is a graph of a typical fluorescence response curve of a group probes illustrative of ratiometric properties. Table 1 is shown illustrating of ideal properties of lead sensors for use in imaging lead levels in environmental testing. The properties listed are—Change in Fluorescence Upon Lead Binding; Absorption Wavelength; Emission Wavelength; Affinity For Lead; Reversible Response; Solubility; Selectively; Biovailability; Toxicity; and Stability. In the column next to the properties are the defined characteristics. The next column indicates whether the fluorescence chemosensor probe listed meets the desired characteristic parameters of the ideal properties.

Referring to FIG. 7, an illustration of a Flow injection system is shown. An alternative application of the present technique is the detection of the presence of lead in a blood sample utilizing a Flow Injection System (FIS). The FIS can be composed of the following components:

Carrier Buffer 702—the carrier buffer is drawn continuously from a container and is primarily responsible for moving the sample through the system and secondarily responsible for establishing a suitable pH for the CL reaction to take place and scavenging contaminants.

Injection Port 704—the sample is introduced to the carrier buffer by means of direct injection through this port.

Pump 1 706—A pump mechanism will be utilized to pull carrier buffer and the sample into the system, then push the sample past the photomultiplier and into the waste container.

Oxidizer Solution 708—The oxidizer solution is a necessary component of the CL reaction and is stored in it own containment.

CL Solution 710—The CL solution is the CL compound used in the analysis which is stored in it own containment.

Pump 2 712—A second pump mechanism will be utilized to pull oxidizer solution and CL solution into the system, then push the sample past the photomultiplier and into the waste container.

Regulator/Mixer 714—This device regulates pressure and flow at the site where the carrier stream meets the reactant stream to ensure proper mixing and safe management of pressure in the tubing.

Presentation Vessel 716—This vessel allows the sample, combined with the reactants, to pool in view of the photomultiplier. Measurement of the luminescence occurs at this point in the system.

Photomultiplier 718—The photomultiplier is device used for detection of photons which works by converting photons to an electrical signal that can be interpreted and recorded.

Recording Device 720—The recording device is capable of interpreting and storing signals from the photomultiplier.

Each of the above components can be connected via tubing with the exception of the recording device which can be connected electronically. Analysis of the output in conjunction with specially tailored standards can be used to determine the lead concentration in media such as blood and environmental samples.

One of the hallmarks of CL reactions is that they proceed very quickly (in the absence of reaction stabilizers) and thus recording images and making measurements on a surface or a sample can be very difficult. To achieve consistency in lead measurement a light-sensitive film (such as Kodak BioMax Film) can be used to capture light emission from surfaces and samples. Similar methods can be used in (at least) the molecular biology field most prominently in applications where CL is being used in place of radioactive labels (Western Blots in particular). The general use of this method is described as follows:

The sample prepared for analysis can be an addition of the CL reactants, the CL compound and an oxidizer. As the reaction proceeds and luminescence is achieved a specially designed cassette containing light-sensitive film can be placed over the sample and a shutter can be opened exposing the film to the luminescence for an appropriate time period depending on the exposure rate and the ambient temperature. The shutter can then closed to protect the film from further exposure and the film is developed and fixed by immersion in the proper solutions. The “fixed” image on the film can then be digitally scanned and analyzed using raster analysis with the ratio of dark and light zones corresponding to the intensity of the CL reaction on the sample.

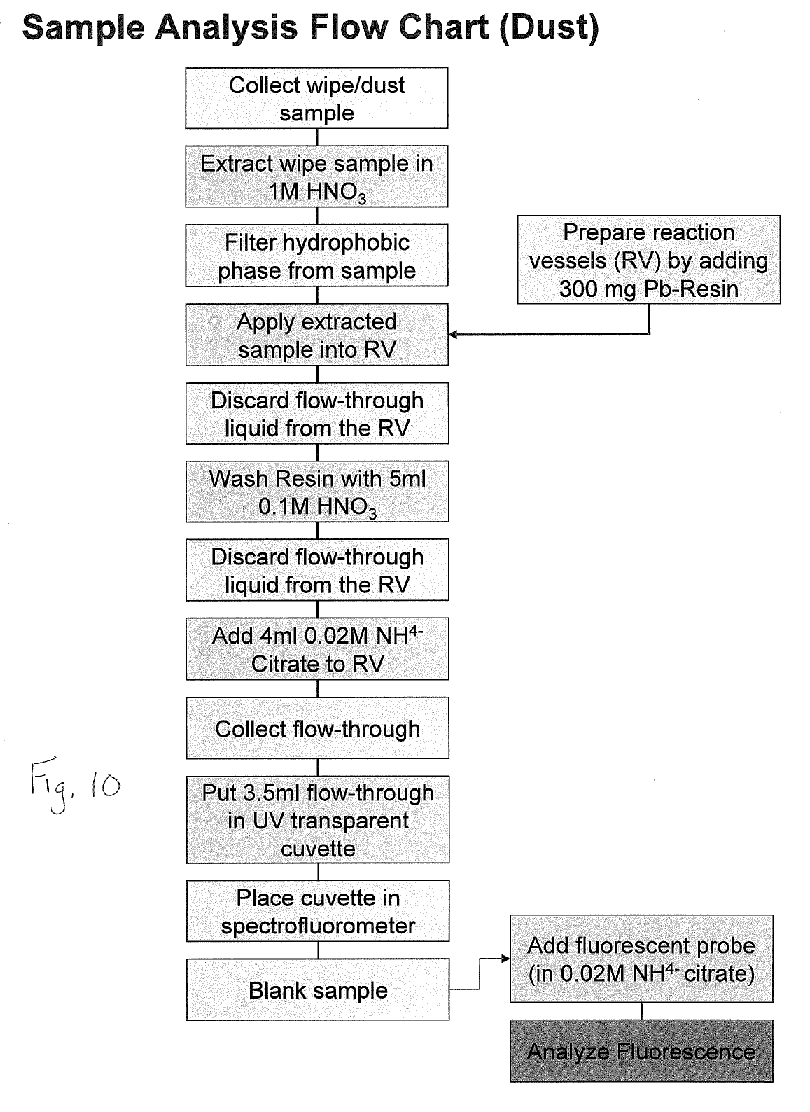

A flow chart of an alternative embodiment of the present invention diagramming the steps for analyzing a dust sample is provided in FIG. 10. First, a sample is collected. This sample can be collected onto a dust wipe, can be collected as pure dust, or can be collected as a solid sample. The sample is then ashed (not required for a pure dust sample) in a muffle furnace. The dust/ash is extracted into 1-Molar Nitric Acid (HNO3). The hydrophobic phase is removed, and the hydrophilic (lead-containing) phase is passed through about approximately 0.3 mg (+/−0.01 mg) of the ion exchange resin. The flow through is discarded as waste, the column is flushed with about approximately 5 mL of 0.1M HNO3, and waste is once again discarded. About approximately 4 mL of 0.02M ammonium citrate is passes through the column and collected. About approximately 3.5 mL of the purified sample is transferred to a UV transparent cuvette and a sample blank reading is recorded. The fluorescent probe (in 0.02M ammonium citrate) is added to the sample. Fluorescence is analyzed and compared to standards. The specific quantities can vary without departing from the scope of the invention.

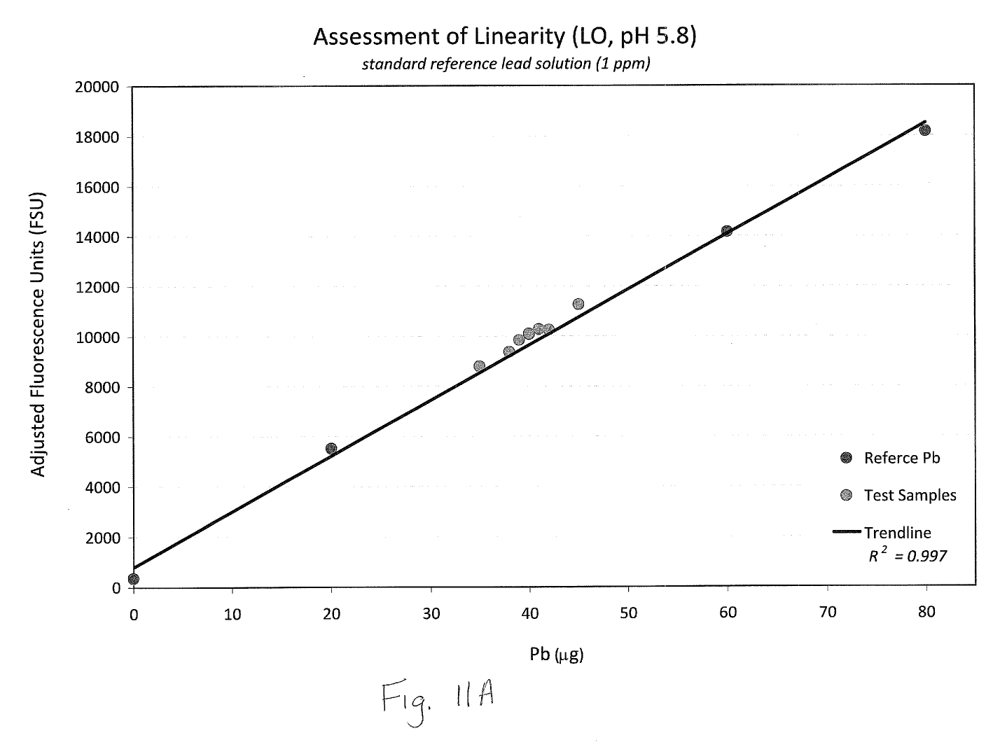

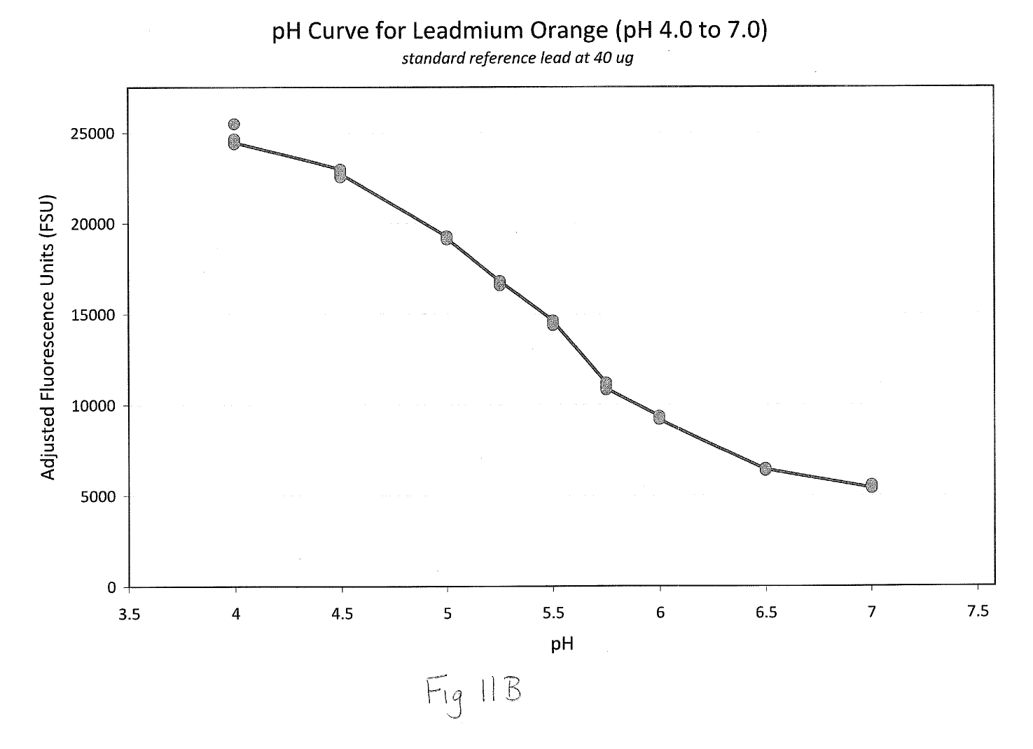

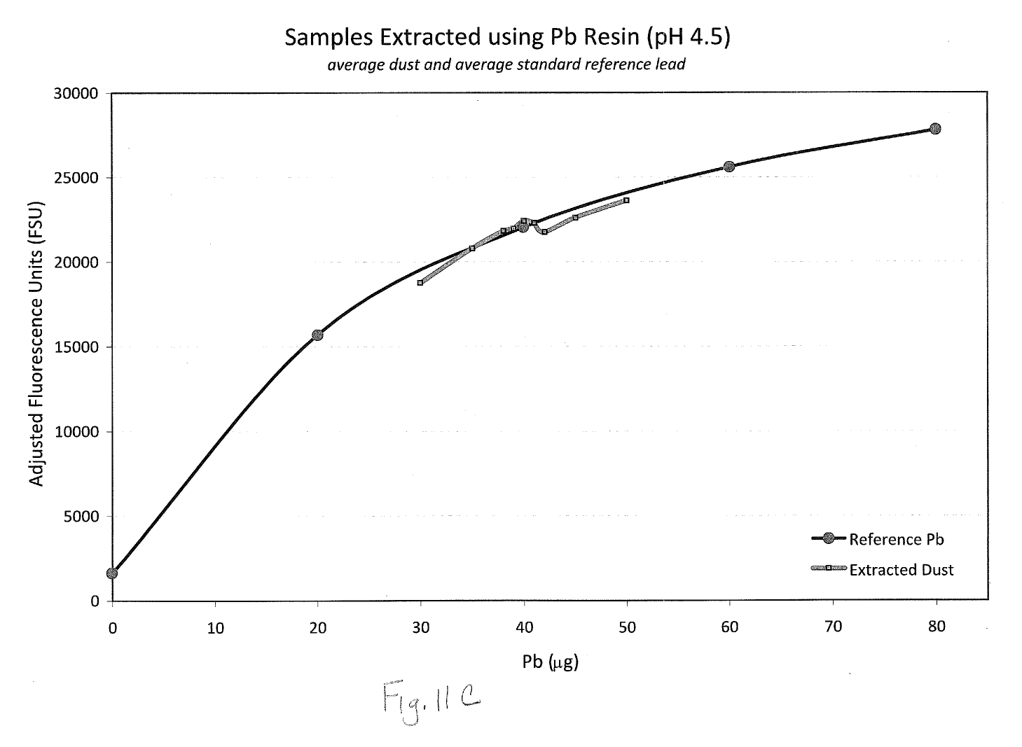

Referring to FIGS. 11A-11C, graphs of experimental data is provided showing the ratiometric relationship attained between lead concentration and fluorescence by our chemifluorescent compound (using ledmium orange). The data shows that there is a near-linear (or at least a ratiometric) relationship between the amount of lead in a sample and the intensity of the fluorescent response. Additionally, this data is important because it shows that the setup is capable of determining lead concentration with a high degree of specificity at regulatory lead levels, as well as over a range of concentrations both higher and lower than current HUD regulatory standards.

TABLE 1
Ideal properties of lead sensors for use in imaging lead levels
in environmental testing (e.g. detection of lead in dust)

| | Sensor for Environmental | |
|---|---|---|
| Ideal Properties of: | Lead (e.g. Dust) | Probe X |
| Change in Fluorescence Upon | Turn on | |
| Lead Binding | | |
| Absorption Wavelength | >280 nm | |
| Emission Wavelength | >400 nm | |
| Affinity for Lead | Kd 10-3-10-6 | |
| Reversible response | N.A. | |
| Solubility | Water solubility may | |
| | be helpful | |
| Selectivity | Pb >> Zn, Ti | |
| Bioavailability | N.A. | |
| Toxicity | Non-toxic if consumers | |
| | are using | |
| Stability | Resistant to degradation | |
| | in the environment | |

N.A. = not applicable

### APPENDIX

### A1. Common Fluorophores

1,5 IAEDANS, 1,8-ANS, 4-Methylumbelliferone, 5-carboxy-2,7 dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-Hydroxy Tryptamine (HAT), 5-ROX (carboxy-X-rhodamine), 5-TAMRA (5-Carboxytetramethylrhodamine), 6-Carboxyrhodamine 6G, 6-CR 6G, 6-JOE, 7-Amino-4-methylcoumarin, 7-Aminoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine variants, Acriflavin, Acriflavin Feulgen SITSA, Aequorin (Photoprotein), AFPs—AutoFluorescent Protein—(Quantum Biotechnologies) see sgGFP, sgBFP, Alexa Fluor, Alizarin Complexon, Alizarin Red, Allophycocyanin (APC), AMC, AMCA-S, AMCA (Aminomethylcoumarin), AMCA-X

Aminoactinomycin D, Aminocoumarin, Aminomethylcoumarin (AMCA), Anilin Blue, Anthrocyl stearate, APC (Allophycocyanin), APC-Cy7, APTRA-BTC, APTS, Astrazon, Brilliant Red 4G, Astrazon Orange R, Astrazon Red 6B, Astrazon Yellow 7 GLL, Atabrine, ATTO-TAG CBQCA, ATTO-TAG FQ, Auramine, Aurophosphine G, Aurophosphine, BAO 9 (Bisaminophenyloxadiazole), BCECF (high pH), BCECF (low pH), Berberine Sulphate, Beta Lactamase, BFP blue shifted GFP (Y66H), Blue Fluorescent Protein, BFP/GFP FRET, Bimane, Bisbenzamide, Bisbenzimide (Hoechst), bis-BTC, Blancophor FFG, Blancophor SV, BOBO-1, BOBO-3, Bodipy variants, BO-PRO-1, BO-PRO-3, Brilliant Sulphoflavin FF, BTC—Ratio Dye Ca2+, BTC-5N, Calcein, Calcein Blue, Calcium Crimson, Calcium Green variants, Calcium Orange, Calcofluor White, Carboxy-X-rhodamine (5-ROX), Cascade Blue, Cascade Yellow, Catecholamine, CCF2 (GeneBlazer), CFDA, CFP—Cyan Fluorescent Protein, CFPNFP FRET, Chlorophyll, Chromomycin A, CL-NERF, CMFDA, Coelenterazine variants, Coumarin Phalloidin, C-phycocyanine, CPM Methylcoumarin, CTC, CTC Formazan, Cy variants, Cyan GFP, cyclic AMP Fluorosensor (FiCRhR), CyQuant Cell Proliferation Assay, Dabcyl, Dansyl variants, DAPI, Dapoxyl variants, DCFDA, DCFH, Dichlorodihydrofluorescein Diacetate), DDAO, DHR (Dihydrorhodamine 123), Di-4-ANEPPS, Di-8-ANEPPS (non-ratio), DiA (4-Di-16-ASP), Dichlorodihydrofluorescein Diacetate (DCFH), DiD variants, Dihydrorhodamine 123 (DHR), DiI (DiIC18(3)), Dinitrophenol, DiO (DiOC18(3)), DiR, DiR (DiIC18(7)), DM-NERF (high pH), DNP, Dopamine, DsRed, DTAF, DY-630-NHS, DY-635-NHS, EBFP, ECFP, EGFP, ELF 97, Eosin, Erythrosin, Erythrosin ITC, Ethidium Bromide, Ethidium homodimer-1 (EthD-1), Euchrysin, EukoLight, Europium (III) chloride, EYFP, Fast Blue, FDA, Feulgen (Pararosaniline), FIF, FITC, FITC Antibody, Flazo Orange, Fluo-3, Fluo-4, Fluorescein (FITC), Fluorescein Diacetate, Fluoro-Emerald, Fluoro-Gold (Hydroxystilbamidine), Fluor-Ruby, Fluor X, FM 1-43, FM 446, Fura variants, Genacryl variants, GeneBlazer (CCF2), GFP variants, Gloxalic Acid, Granular Blue, Haematoporphyrin, Hoechst variants, HPTS, Hydroxycoumarin, Hydroxystilbamidine (FluoroGold), Hydroxytryptamine, Indo-1, Indodicarbocyanine (DiD), Indotricarbocyanine (DiR), Intrawhite Cf, JC-1, JO-JO-1, JO-PRO-1, LaserPro, Laurodan, LDS 751 (DNA), LDS 751 (RNA), Leucophor PAF, Leucophor SF, Leucophor WS, Lissamine Rhodamine, Lissamine Rhodamine B, LIVE/DEAD Kit Animal Cells, Calcein/Ethidium homodimer, LOLO-1, LO-PRO-1, Lucifer Yellow, Lyso Tracker variants, LysoSensor variants, Mag Green, Magdala Red (Phloxin B), Mag-Fura variants, Mag-Indo-1, Magnesium Green, Magnesium Orange, Malachite Green, Marina Blue, Maxilon Brilliant Flavin 10 GFF, Maxilon Brilliant Flavin 8 GFF, Merocyanin, Methoxycoumarin, Mitotracker variants, Mitramycin, Monobromobimane, Monobromobimane (mBBr-GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant, lavin E8G, Oregon Green variants, Pacific Blue, Pararosaniline (Feulgen), PBFI, PE-Cy5, PE-Cy7, PerCP, PerCP-Cy5.5, PE-TexasRed [Red 613], Phloxin B (Magdala Red), Phorwite variants, Phosphine 3R, PhotoResist, Phycoerythrin B [PE], Phycoerythrin R [PE], PKH26 (Sigma), PKH67, PMIA, Pontochrome Blue Black, POPO-1, POPO-3, PO-PRO-1, PO-PRO-3, Primuline, Procion Yellow, Propidium Iodid (Pi), PyMPO, Pyrene, Pyronine, Pyronine B, Pyrozal Brilliant Flavin 7GF, QSY 7, Quinacrine Mustard, Red 613 [PE-TexasRed], Resorufin, RH 414, Rhod-2, Rhodamine variants, Rose Bengal, R-phycocyanine, R-phycoerythrin (PE), rsGFP, S65A, S65C, S65L, S65T, Sapphire GFP, SBFI, Serotonin, Sevron Brilliant Red 2B, Sevron variants, sgBFP, sgBFP (super glow BFP), sgGFP, sgGFP (super glow GFP), SITS, SITS (Primuline), SITS (Stilbene isothiosulphonic Acid), SNAFL calcein, SNAFL-1, SNAFL-2, SNARF calcein, SNARF1, Sodium Green, SpectrumAqua, SpectrumGreen, SpectrumOrange, Spectrum Red, SPQ (6-methoxy-N-(3-sulfopropyl)quinolinium), Stilbene, Sulphorhodamine B can C, Sulphorhodamine G Extra, SYTO variants, SYTOX variants, Tetracycline, Tetramethylrhodamine (TRITC), Texas Red, Texas Red-X conjugate, Thiadicarbocyanine (DiSC3), Thiazine Red R, Thiazole Orange, Thioflavin variants, Thiolyte, Thiozole Orange, Tinopol CBS (Calcofluor White), TMR, TO-PRO variants, TOTO-1, TOTO-3, TriColor (PE-Cy5), TRITC, TetramethylRodaminelsoThioCyanate, True Blue, TruRed, Ultralite, Uranine B, Uvitex SFC, wt GFP, WW 781, X-Rhodamine, XRITC, Xylene Orange, Y66F, Y66H, Y66W, Yellow GFP, YFP, YO-PRO-1, YO-PRO-3, YOYO-1, YOYO-3

### APPENDIX

### A2. Common Oxidizers

Bleach, Nitrites Bromates, Nitrous oxide, Bromine, Ozanates, Butadiene, Oxides, Chlorates, Oxygen, Chloric Acid, Oxygen difluoride, Chlorine, Ozone Chlorite, Peracetic Acid, Chromates, Perhaloate, Chromic Acid, Perborates, Dichromates, Percarbonates, Fluorine, Perchlorates, Haloate, Perchloric Acid, Halogens, Permanganates, Hydrogen Peroxide, Peroxides, Hypochlorites, Persulfate Iodates, Sodium Borate, Perhydrate, Mineral Acid, Sulfuric Acid, Nitrates, Nitric Acid

The various fluorogenic probe lead detection examples shown above illustrate novel, portable, fast and cost efficient method for detecting lead in a residential environment. A user of the present invention may choose any of the above fluorogenic probe detection embodiments, or an equivalent thereof, depending upon the desired application. In this regard, it is recognized that various forms of the subject fluorogenic probe invention could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present invention.

Other aspects, objects and advantages of the present invention can be obtained from a study of the drawings, the disclosure and the appended claims.

What is claimed is:

1. A method for using fluorogenic chemosensor probes for detection of lead for lead abatement activities comprising the steps of:
wiping a suspect surface with a wipe;
placing the wipe in a developer solution with a fluorogenic chemosensor probe and agitating said wipe in solution to assist in the extraction of any embedded matter resulting from the swipe;
capturing an image of the solution; and
screening the image for fluorescence emissions indicative of the presence of lead.

2. The method for using a fluorogenic chemosensor probe as recited in claim 1, where the developer solution is a chemically compatible buffer solution with the fluorogenic chemosensor probe selected from a group of probes consisting of those described in Appendix A1.

3. The method for using a fluorogenic chemosensor probe as recited in claim 1, where the chemically compatible buffer solution is a resin based acid extraction.

4. The method for using a fluorogenic chemosensor probe as recited in claim 1, where capturing an image is capturing a digital image.

5. The method for using a fluorogenic chemosensor probe as recited in claim 1, where the step of screening the image includes processing the image with software operable to correlate lead concentration with average fluorescence intensity.

6. A method for using fluorogenic chemosensor probes for detection of lead for lead abatement activities comprising the steps of:
wiping a suspect surface with a wipe;
placing the wipe in a developer solution with a fluorogenic chemosensor probe and agitating said wipe in solution to assist in the extraction of any embedded matter resulting from the swipe;
illuminating the solution and capturing an image of the solution; and
screening the image for fluorescence emissions indicative of the presence of lead.

7. The method for using a fluorogenic chemosensor probe as recited in claim 6, where the developer solution is a chemically compatible buffer solution with the fluorogenic chemosensor probe selected from a group of probes consisting of those described in Appendix A1.

8. The method for using a fluorogenic chemosensor probe as recited in claim 6, where the chemically compatible buffer solution is a resin based acid extraction.

9. The method for using a fluorogenic chemosensor probe as recited in claim 6, where illuminating the solution is illuminating with a light selected from a group of lights consisting of ultraviolet lights, black lights and filtered visible lights.

10. The method for using a fluorogenic chemosensor probe as recited in claim 6, where the step of screening the image includes processing the image with software operable to correlate lead concentration with average fluorescence intensity.

11. A method for using fluorogenic chemosensor probes for detection of lead for lead abatement activities comprising the steps of:
spraying a suspect surface with a developer solution containing a fluorogenic chemosensor probe;
illuminating the suspect surface and capturing an image of the suspect surface ; and
screening the image for fluorescence emissions indicative of the presence of lead.

12. The method for using a fluorogenic chemosensor probe as recited in claim 11, where the step of spraying is performed with a sprayer selected from a group sprayers consisting of a spray bottle and a powered spraying device.

13. The method for using a fluorogenic chemosensor probe as recited in claim 11, where the developer solution comprises a chemically compatible buffer solution with a fluorogenic chemosensor probe selected from a group of probes consisting of those described in Appendix A1.

14. The method for using a fluorogenic chemosensor probe as recited in claim 11, where the chemically compatible buffer solution is a resin based acid extraction.

15. The method for using a fluorogenic chemosensor probe as recited in claim 11, where the step of screening the image includes processing the image with software operable to correlate lead concentration with average fluorescence intensity.

16. A method for using fluorogenic chemosensor probes for detection of lead for lead abatement activities comprising the steps of:
placing a sticky side of a sticky back paper wipe against a suspect surface where said sticky side has imbedded therein a fluorescence chemosensor probe;
peeling the sticky back paper wipe off the suspect surface;
illuminating the sticky side and capturing an image of the sticky side; and
screening the image for fluorescence emissions indicative of the presence of lead.

17. The method for using a fluorogenic chemosensor probe as recited in claim 16, where the sticky side is imbedded with at least one of the material products selected from a group of products consisting of low tack and medium tack adhesive.

18. The method for using a fluorogenic chemosensor probe as recited in claim 17, where the imbedded fluorescence chemosensor probe is selected from a group of probes consisting of those described in Appendix A1.

19. The method for using a fluorogenic chemosensor probe as recited in claim 16, where the step of illuminating is with a light selected from a group of lights consisting of ultra violet lights, black lights and filtered visible lights.

20. The method for using a fluorogenic chemosensor probe as recited in claim 16, where the step of capturing an image is capturing a digital image and the step of screening the image is performed with a computerized signal processing device

21. A method for using fluorogenic chemosensor probes for detection of lead for lead abatement activities comprising the steps of:
placing a sticky side of a sticky back paper wipe against a suspect surface;
peeling the sticky back paper wipe off the suspect surface;
placing the wipe in a developer solution with a fluorogenic chemosensor probe and agitating said wipe in solution to assist in the extraction of any embedded matter resulting from the swipe;
capturing an image of the solution; and
screening the image for fluorescence emissions indicative of the presence of lead.

22. The method for using a fluorogenic chemosensor probe as recited in claim 21, where the sticky side is imbedded with at least one of the material products selected from a group of products consisting of low tack and medium tack adhesive.

23. The method for using a fluorogenic chemosensor probe as recited in claim 22, where the developer solution is a chemically compatible buffer solution with the fluorogenic chemosensor probe selected from a group of probes consisting of those described in Appendix A1.

24. The method for using a fluorogenic chemosensor probe as recited in claim 21, further comprising the step of:
illuminating the solution where illuminating is with a light selected from a group of lights consisting of ultraviolet lights, black lights and filtered visible lights.

25. The method for using a fluorogenic chemosensor probe as recited in claim 21, where the step of screening the image includes processing the image with software operable to correlate lead concentration with average fluorescence intensity.

26. A method for using chemifluorescent probes for detection of lead for lead abatement activities comprising the steps of:
wiping a suspect surface with a wipe;
placing the wipe in an extraction solution with agitation of said solution followed by neutralization of the solution;
the addition of a chemifluorescent probe;
analyzing the fluorescence intensity of the solution; and
determining whether the sample meets clearance standards.

27. The method for using a chemifluorescent probe as recited in claim 26, where the extraction solution is capable of solvating lead from house dust with agitation.

28. The method for using a chemifluorescent probe as recited in claim 26, where neutralization makes the solution suitable for the addition of the chemifluorescent probe.

29. The method for using a chemifluorescent probe as recited in claim 26, where the step of adding the chemifluorescent probe performed with the chemifluorescent probe selected from a group of probes consisting of those listed in Appendix A1.

30. The method for using a chemifluorescent probe as recited in claim 26, where florescence is measured in the field using a portable spectrofluorimeter or in a laboratory.

31. The method for using a chemifluorescent probe as recited in claim 26, where the step of determining clearance based extrapolation from a standard curve.

32. A method for using chemifluorescent probes for detection of lead for lead abatement activities comprising the steps of:
wiping a suspect surface with a wipe;
applying a developer solution to the wipe which contains a chemifluorescent probe;
illuminating the wipe using a light source which emits a wavelength appropriate for excitation of the chemifluorescent probe employed;
capturing an image of the wipe; and
screening the image for fluorescence emissions indicative of the presence of lead.

33. The method for using a chemifluorescent probe as recited in claim 32, where the developer solution contains the chemifluorescent probe selected from a group of probes consisting of those listed in Appendix A1 and is capable of solvating lead from house dust on the wipe.

34. The method for using a chemifluorescent probe as recited in claim 32, where a light source is used which emits a wavelength appropriate for excitation of the chemifluorescent probe employed.

35. The method for using a chemifluorescent probe as recited in claim 32, where an image of the wipe is recorded using a camera with appropriate illumination or through an imaging system that can scan the wipe and generate an image

36. The method for using a chemifluorescent probe as recited in claim 32, where the step of screening the image includes processing the image with software to correlate lead concentration with average fluorescence intensity by utilizing such methods as raster image analysis.

37. A method for using chemifluorescent probes for detection of lead for lead abatement activities comprising the steps of:
spraying a suspect surface with a developer solution containing a chemifluorescent probe;
illuminating the suspect surface and capturing an image of the suspect surface ; and
screening the image for fluorescence emissions indicative of the presence of lead.

38. The method for using a chemifluorescent probe as recited in claim 37, where the step of spraying is performed with any sprayer capable of delivering an even and diffuse quantity of the solution to the surface.

39. The method for using a chemifluorescent probe as recited in claim 37, where the developer solution contains the chemifluorescent probe selected from a group of probes consisting of those listed in Appendix A1 and is capable of solvating lead from house dust on the surface.

40. The method for using a chemifluorescent probe as recited in claim 37, where a light source is used which emits a wavelength appropriate for excitation of the chemifluorescent probe employed.

41. The method for using a chemifluorescent probe as recited in claim 37, where an image of the surface is recorded using a camera with appropriate illumination or through an imaging system that can scan the surface and generate an image

42. The method for using a chemifluorescent probe as recited in claim 37, where the step of screening the image includes processing the image with software to correlate lead concentration with average fluorescence intensity by utilizing such methods as raster image analysis.

43. A method for using chemifluorescent probes for detection of lead for lead abatement activities comprising the steps of:
spraying a suspect surface with a cleaning solution comprised of detergent and a chemifluorescent probe;
illuminating the suspect surface; and
using the extent and location of fluorescence to inform where cleaning efforts should be concentrated.

44. The method for using a chemifluorescent probe as recited in claim 43, where the step of spraying is performed with any sprayer capable of delivering an even and diffuse quantity of the solution to the surface.

45. The method for using a chemifluorescent probe as recited in claim 43, where the cleaning solution contains the chemifluorescent probe selected from a group of probes consisting of those listed in Appendix A1 and a detergent capable of solvating lead and house dust from the surface.

46. The method for using a chemifluorescent probe as recited in claim 43, where a light source is used which emits a wavelength appropriate for excitation of the chemifluorescent probe employed.

47. The method for using a chemifluorescent probe as recited in claim 43, where the extent and location of fluorescence inform abatement specialists where cleaning efforts should be concentrated

48. A method for using chemifluorescent probes for detection of lead for lead abatement activities comprising the steps of:
pressing a paper or cloth material coated on one side with low tack adhesive evenly against the surface to be tested;
peeling the lift sample material off the suspect surface;
applying a developer solution to the lift sample which contains a chemifluorescent probe;
illuminating the lift sample using a light source which emits a wavelength appropriate for excitation of the chemifluorescent probe employed;
capturing an image of the sample; and
screening the image for fluorescence emissions indicative of the presence of lead.

49. The method for using a chemifluorescent probe as recited in claim 48, where the sticky side of the paper or cloth material is imbedded with a low tack adhesive capable of capturing dust particles in which the sample can be easily released from the surface without causing damage. Optionally the material comprising the lift sample can be soluble in the extraction solution.

50. The method for using a chemifluorescent probe as recited in claim 48, where the developer solution contains the chemifluorescent probe selected from a group of probes consisting of those listed in Appendix A1 and is capable of solvating lead from house dust on the lift sample.

51. The method for using a chemifluorescent probe as recited in claim 48, where a light source is used which emits a wavelength appropriate for excitation of the chemifluorescent probe employed.

52. The method for using a chemifluorescent probe as recited in claim 48, where an image of the lift sample is recorded using a camera with appropriate illumination or through an imaging system that can scan the lift sample and generate an image

53. The method for using a chemifluorescent probe as recited in claim 48, where the step of screening the image includes processing the image with software to correlate lead concentration with average fluorescence intensity by utilizing such methods as raster image analysis.

54. A method for using chemifluorescent probes for detection of lead for lead abatement activities comprising the steps of:
vacuuming a carpeted, cloth, or hard surface with a vacuum sampler;
placing the dust sample in an extraction solution with agitation of said solution followed by neutralization of the solution;
the addition of a chemifluorescent probe;
analyzing the fluorescence intensity of the solution; and
determining whether the sample meets clearance standards.

55. The method for using a chemifluorescent probe as recited in claim 54, where the extraction solution is capable of solvating lead from house dust with adgitation.

56. The method for using a chemifluorescent probe as recited in claim 54, where neutralization makes the solution suitable for the addition of the chemifluorescent probe.

57. The method for using a chemifluorescent probe as recited in claim 54, where the step of adding the chemifluorescent probe performed with the chemifluorescent probe selected from a group of probes consisting of those listed in Appendix A1.

58. The method for using a chemifluorescent probe as recited in claim 54, where florescence is measured in the field using a portable spectrofluorimeter or in a laboratory.

59. The method for using a chemifluorescent probe as recited in claim 54, where the step of determining clearance based extrapolation from a standard curve.

60. A method for using chemiluminescent probes for detection of lead for lead abatement activities comprising the steps of:
wiping a suspect surface with a wipe;
placing the wipe in an extraction solution with agitation of said solution followed by neutralization of the solution;
the addition of a chemiluminescent probe;
analyzing the luminescent intensity of the solution; and
determining whether the sample meets clearance standards.

61. The method for using a chemiluminescent probe as recited in claim 60, where the extraction solution is capable of solvating lead from house dust with agitation.

62. The method for using a chemiluminescent probe as recited in claim 60, where neutralization makes the solution suitable for the addition of the chemiluminescent probe.

63. The method for using a chemiluminescent probe as recited in claim 60, where the step of adding the chemiluminescent probe is performed using a chemiluminescent probe selected from a group of probes consisting of those listed in Appendix A1.

64. The method for using a chemiluminescent probe as recited in claim 60, where luminescence is measured in the field using a portable luminometer or in a laboratory.

65. The method for using a chemiluminescent probe as recited in claim 60, where the step of determining clearance based extrapolation from a standard curve.

66. A method for using chemiluminescent probes for detection of lead for lead abatement activities comprising the steps of:
wiping a suspect surface with a wipe;
applying a developer solution to the wipe which contains a chemiluminescent probe;
placing the activated wipe in a dark box;
capturing an image of the wipe; and
screening the image for luminescence indicative of the presence of lead.

67. The method for using a chemiluminescent probe as recited in claim 66, where the developer solution contains the chemiluminescent probe selected from a group of probes consisting of those listed in Appendix A1 and is capable of solvating lead from house dust on the wipe.

68. The method for using a chemiluminescent probe as recited in claim 66, where dark box is used to eliminate background light.

69. The method for using a chemiluminescent probe as recited in claim 66, where an image of the wipe is recorded using a camera or through an imaging system that can scan the wipe and generate an image

70. The method for using a chemiluminescent probe as recited in claim 66, where the step of screening the image includes processing the image with software to correlate lead concentration with average luminescence by utilizing such methods as raster image analysis.

71. A method for using chemiluminescent probes for detection of lead for lead abatement activities comprising the steps of:
spraying a suspect surface with a developer solution containing a chemiluminescent probe;
darkening the suspect surface and capturing an image of the suspect surface ; and
screening the image for luminescence indicative of the presence of lead.

72. The method for using a chemiluminescent probe as recited in claim 71, where the step of spraying is performed with any sprayer capable of delivering an even and diffuse quantity of the solution to the surface.

73. The method for using a chemiluminescent probe as recited in claim 71, where the developer solution contains the chemiluminescent probe selected from a group of probes consisting of those listed in Appendix A1 and is capable of solvating lead from house dust on the surface.

74. The method for using a chemiluminescent probe as recited in claim 71, where a dark box is used to eliminate background light.

75. The method for using a chemiluminescent probe as recited in claim 71, where an image of the surface is recorded using a camera or through an imaging system that can scan the surface and generate an image

76. The method for using a chemiluminescent probe as recited in claim 71, where the step of screening the image includes processing the image with software to correlate lead concentration with average luminescence by utilizing such methods as raster image analysis.

77. A method for using chemiluminescent probes for detection of lead for lead abatement activities comprising the steps of:
spraying a suspect surface with a cleaning solution comprised of detergent and a chemiluminescent probe; and
using the extent and location of fluorescence to inform where cleaning efforts should be concentrated.

78. The method for using a chemiluminescent probe as recited in claim 77, where the step of spraying is performed with any sprayer capable of delivering an even and diffuse quantity of the solution to the surface.

79. The method for using a chemiluminescent probe as recited in claim 77, where the cleaning solution contains the chemiluminescent probe selected from a group of probes consisting of those listed in Appendix A1 and a detergent capable of solvating lead and house dust from the surface.

80. The method for using a chemiluminescent probe as recited in claim 77, where the extent and location of luminescence informs abatement specialists where cleaning efforts should be concentrated

81. A method for using chemiluminescent probes for detection of lead for lead abatement activities comprising the steps of:
pressing a paper or cloth material coated on one side with low tack adhesive evenly against the surface to be tested;
peeling the lift sample material off the suspect surface;
applying a developer solution to the lift sample which contains a chemiluminescent probe;
placing the activated wipe in a dark box;
capturing an image of the sample; and
screening the image for luminescence indicative of the presence of lead.

82. The method for using a chemiluminescent probe as recited in claim 81, where the sticky side of the paper or cloth material is imbedded with a low tack adhesive capable of capturing dust particles in which the sample can be easily released from the surface without causing damage. Optionally the material comprising the lift sample can be soluble in the extraction solution.

83. The method for using a chemiluminescent probe as recited in claim 81, where the developer solution contains the chemiluminescent probe selected from a group of probes consisting of those listed in Appendix A1 and is capable of solvating lead from house dust on the lift sample.

84. The method for using a chemiluminescent probe as recited in claim 81, where dark box is used to eliminate background light.

85. The method for using a chemiluminescent probe as recited in claim 81, where an image of the lift sample is recorded using a camera or through an imaging system that can scan the lift sample and generate an image

86. The method for using a chemiluminescent probe as recited in claim 81, where the step of screening the image includes processing the image with software to correlate lead concentration with average luminescence by utilizing such methods as raster image analysis.

87. A method for using chemiluminescent probes for detection of lead for lead abatement activities comprising the steps of:
vacuuming a carpeted, cloth, or hard surface with a vacuum sampler;
placing the dust sample in an extraction solution with agitation of said solution followed by neutralization of the solution;
the addition of a chemiluminescent probe;
analyzing the luminescence of the solution; and
determining whether the sample meets clearance standards.

88. The method for using a chemiluminescent probe as recited in claim 87, where the extraction solution is capable of solvating lead from house dust with agitation.

89. The method for using a chemiluminescent probe as recited in claim 87, where neutralization makes the solution suitable for the addition of the chemiluminescent probe.

90. The method for using a chemiluminescent probe as recited in claim 87, where florescence is measured in the field using a portable luminometer or in a laboratory.

91. The method for using a chemiluminescent probe as recited in claim 87, where the step of determining clearance based extrapolation from a standard curve.

92. A method for using fluorogenic chemosensor probes for detection of lead for lead abatement activities comprising the steps of:
wiping a suspect surface with a wipe collecting a sample;
extracting the sample in 1-Molar Nitric Acid (HNO3);
filtering the hydrophobic phase from the sample by preparing a reaction vessel by adding 300 mg Pb-Resin and applying the extracted sample into the reaction vessel;
passing the hydrophilic (lead-containing) phase is through 0.3 mg of the ion exchange resin;
discarding the flow through from the reaction vessel as waste;
flushing the column with 5 mL of 0.1M HNO3;
discarding the waste;
passing through 4 mL of 0.02M ammonium citrate through the column and collecting the flow through;
transferring 3.5 mL of the purified sample to a UV transparent cuvette;
adding the fluorescent probe (in 0.02M ammonium citrate) to the sample; and
analyzing the fluorescence and comparing to standards.
